(19) United States
(12) Patent Application Publication
(10) Pub. No.: US 2025/0073071 A1
(43) Pub. Date: Mar. 6, 2025

(54) METHOD AND DEVICE FOR CORNEAL CROSS-LINKING WITH REAL-TIME MONITORING

(71) Applicant: Alcon Inc., Fribourg (CH)

(72) Inventors: Gangjun LIU, Portland, OR (US); Lingfeng YU, Rancho Santa Margarita, CA (US)

(21) Appl. No.: 18/815,973

(22) Filed: Aug. 27, 2024

Related U.S. Application Data
(60) Provisional application No. 63/579,403, filed on Aug. 29, 2023.

(51) Int. Cl. A61F 9/007; A61F 9/008

(52) U.S. Cl. CPC A61F 9/0079; A61F 2009/00851; A61F 2009/00872

(57) ABSTRACT

A system and method for corneal cross-linking with real-time monitoring are provided. The method comprises determining a plurality of measurement locations in a region of a cornea, and applying a corneal cross-linking treatment to the region of the cornea. During the application of the corneal cross-linking treatment, the method also comprises acquiring a temporal OCT interferogram at each OCT measurement location, generating temporal complex OCT data based on the temporal OCT interferogram, determining biomechanical data based on the temporal complex OCT data, and adjusting the corneal cross-linking treatment based on the biomechanical data.

### CROSS-REFERENCE TO RELATED APPLICATIONS

This application claims the benefit of U.S. Provisional Application Ser. No. 63/579,403 (filed on Aug. 29, 2023), the content of which is incorporated herein by reference in its entirety.

### BACKGROUND

The present disclosure relates to methods and devices for corneal cross-linking with real-time monitoring. More particularly, the present disclosure relates to a corneal biomechanical measurement device for monitoring the corneal cross-linking in real-time.

Corneal cross-linking (also known as corneal collagen cross-linking or CXL) is a medical procedure that is used to treat corneal ectasia caused by certain diseases (such as keratoconus, etc.), complications from surgery (such as LASIK, etc.), as well as other conditions. The corneal stroma is a dense connective tissue that contains organized layers of tightly distributed collagen fibrils that form strong chemical bonds or cross-links between adjacent fibrils. The cross-linked collagen fibrils provide a strong mechanical structure that, inter alia, maintains the proper curvature of the cornea. When cross-links between adjacent collagen fibrils begin to break due to disease, complications from surgery, etc., the organized layers become weakened, the mechanical stability of the cornea is reduced, and the curvature of the cornea begins to deform from a round shape to a conical shape (corneal ectasia).

CXL strengthens the cornea by forming new cross-links between adjacent collagen fibrils, which may halt the progression of keratoconus, recover corneal strength lost by the removal of corneal tissue during LASIK, etc. After the corneal epithelium has been removed and a photosensitizer, such as riboflavin, has been applied to the cornea for about 30 minutes, ultraviolet (UV) light is focused on the cornea for about 30 minutes. The photodynamic interaction between the UV light and the photosensitizer creates reactive oxygen species, which induce the formation of new cross-links between adjacent collagen fibrils to strengthen the cornea. The clinical outcome of CXL on a particular cornea is typically evaluated several weeks after treatment.

### SUMMARY

Embodiments of the present disclosure advantageously provide a system and method for corneal cross linking with real-time monitoring. The method comprises determining a plurality of measurement locations in a region of a cornea, and applying a corneal cross-linking treatment to the cornea. During the application of the corneal cross-linking treatment, the method also comprises measuring corneal the biomechanical properties at each measurement location and adjusting the corneal cross-linking treatment based on the biomechanical data.

### BRIEF DESCRIPTION OF THE DRAWINGS

FIGS. 1A, 1B and 1C depict block diagrams of an example system, in accordance with embodiments of the present disclosure.

FIG. 2 depicts a block diagram of an example OCT engine, in accordance with embodiments of the present disclosure.

FIG. 3A depicts a block diagram of an example control computer, in accordance with embodiments of the present disclosure.

FIG. 3B depicts a block diagram of another example control computer, in accordance with embodiments of the present disclosure.

FIG. 4 depicts a flow diagram illustrating functionality associated with measuring corneal biomechanics, in accordance with embodiments of the present disclosure.

### DETAILED DESCRIPTION

Certain embodiments of the present disclosure advantageously provide a method and integrated CXL and OCT system that applies, monitors and adjusts corneal cross-linking treatment in real-time. The integrated CXL and OCT system advantageously provides high-resolution OCT-based imaging and biomechanical data determination, treatment adjustment based on the biomechanical data, real-time feedback of the effect of the treatment, personalized patient treatment based on the feedback, avoidance of under-treatment such as insufficient UV light illumination or photosensitizer application, avoidance of over-treatment such as excessive UV light illumination or photosensitizer application, as well as other advantages.

In a medical imaging context, an OCT system directs a coherent light beam towards biological tissue, and then measures the interference between a portion of the original coherent light beam and the scattered light reflected back to the OCT system from a particular location on (or within) the biological tissue. The interference is directly related to the reflectivity of the biological tissue at that location. For example, an ocular or ophthalmic OCT system may be used to acquire high-resolution images of the cornea and retina, to determine ocular dimensions, to diagnose various ocular pathologies, etc.

The OCT system generates a one-dimensional “A-scan” by measuring the reflectivity at different depths (axial dimension) at the same location using time domain OCT (TD-OCT) or frequency (Fourier) domain OCT (FD-OCT). The OCT system generates a two-dimensional “B-scan” by combining A-scans acquired at different lateral locations (lateral dimension). The OCT system may also generate an “M-mode” (motion) scan by performing multiple A-scans at the same location, which generates temporal OCT data for that location. Additionally, the OCT system may generate a three-dimensional “C-scan” or volume scan by combining multiple B-scans at different elevations (elevation dimension), which may also include M-mode scans at each location (representing a fourth dimension, time).

Typically, corneal cross-linking treatments last for about an hour, which includes about 30 minutes for photosensitizer soaking and 20-30 minutes of UV light exposure. Acquisition of temporal OCT data at all of the OCT measurement locations in the region of the cornea may require a few seconds. Because OCT systems are non-invasive and offer a non-contact measurement technique, OCT measurements may be performed periodically and/or at will. For example, the OCT measurements may be performed every few minutes during the corneal cross-linking treatment, which allows monitoring of the corneal structure, determination of the corneal biomechanics, and control of the corneal cross-linking treatment in-situ in real-time.

Certain embodiments of the present disclosure provide a method and system for corneal cross-linking with real-time monitoring. Certain embodiments of the method comprise determining a plurality of measurement locations in a region of a cornea, and applying a corneal cross-linking treatment to the region of the cornea. During the application of the corneal cross-linking treatment, the method also comprises acquiring temporal OCT data at each OCT measurement location, generating temporal complex OCT data based on the temporal OCT data, determining biomechanical data based on the temporal complex OCT data, and adjusting the corneal cross-linking treatment based on the biomechanical data.

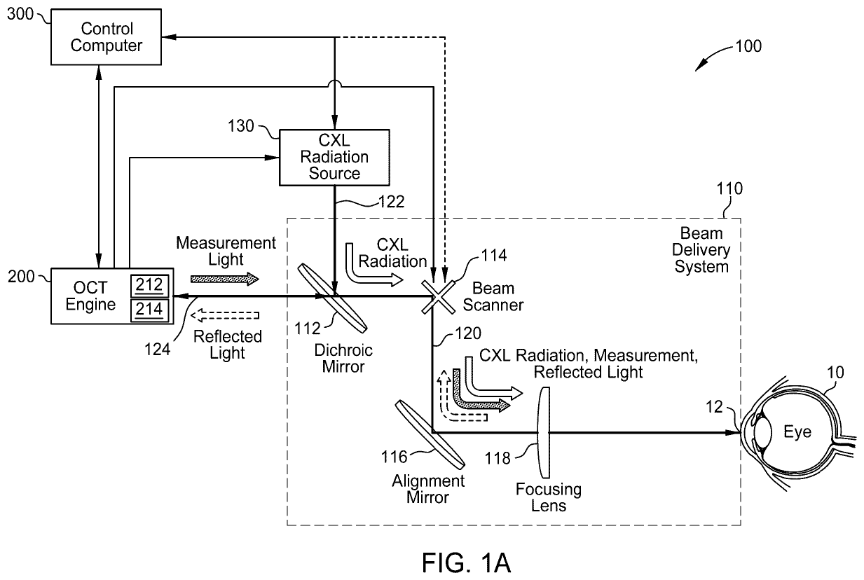

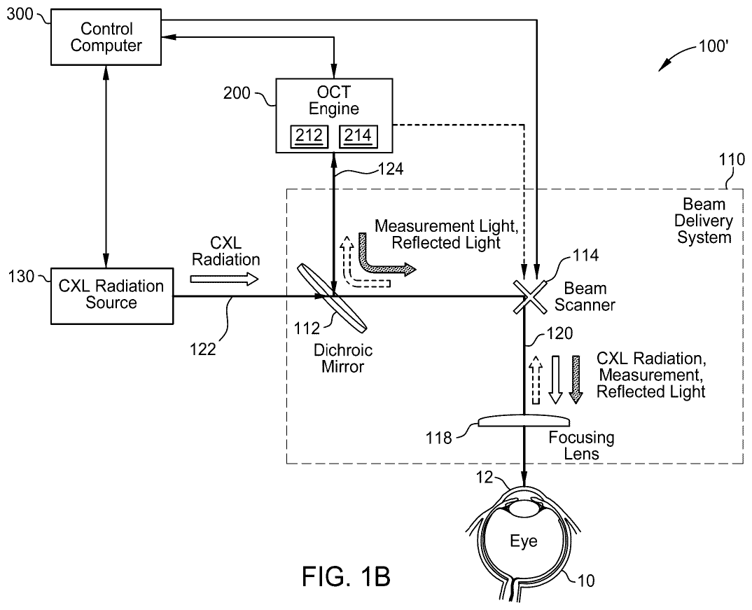

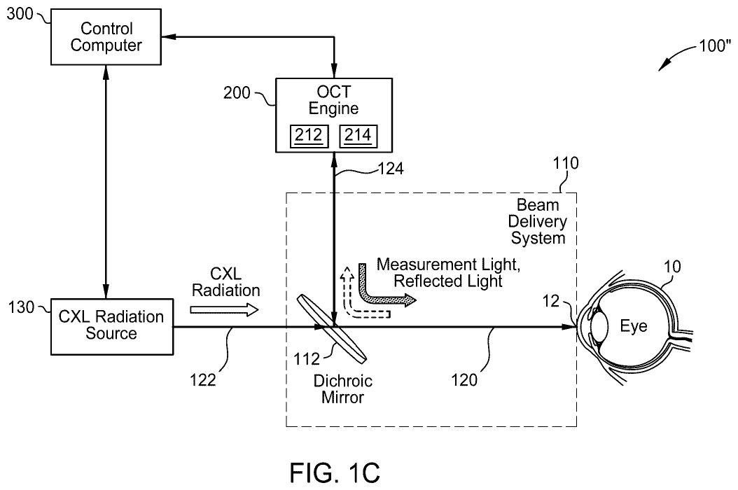

FIG. 1A depicts a block diagram of system 100 for corneal cross-linking with real-time monitoring, in accordance with embodiments of the present disclosure.

In certain embodiments, system 100 includes, inter alia, beam delivery system (BDS) 110, CXL radiation source 130, OCT engine 200 and control computer 300.

In certain embodiments, OCT engine 200 and control computer 300 are separate devices that are communicatively coupled using a wired or wireless connection, such as USB, Ethernet, Bluetooth, WiFi, etc. In certain other embodiments, OCT engine 200 is a component of control computer 300, such as one or more peripheral component interconnect express (PCIe) expansion boards, a PCIe expansion board coupled to one or more external modules, etc. In certain embodiments, OCT engine 200 may be housed within an external PCIe expansion system enclosure coupled to control computer 300 via a PCIe connection.

Generally, BDS 110 is configured to, inter alia, (1) receive, propagate and focus radiation (such as UV light) from CXL radiation source 130 onto cornea 12, (2) receive, propagate and focus low-coherence (LC) light from OCT engine 200 onto cornea 12, and (3) receive, focus and propagate reflected LC light from cornea 12 to OCT engine 200.

In certain embodiments, BDS 110 is a free-space optical system that includes dichroic mirror 112, beam scanner 114, alignment mirror 116, and focusing lens 118 that define common optical path 120. Radiation (such as UV light) emitted from CXL radiation source 130 propagates along optical path 122 to BDS 110, light from LC light source 212 of OCT engine 200 propagates along optical path 124 to BDS 110, and reflected LC light from BDS 110 propagates along optical path 124 to reflected LC light detector 214 of OCT engine 200.

Dichroic mirror 112 is configured to reflect or deflect the radiation (such as UV light) propagating along optical path 122 into common optical path 120, to pass the LC light propagating along optical path 124 into common optical path 120, and to pass the reflected LC light propagating along common optical path 120 into optical path 124. In other words, the radiation propagating along optical path 122 and the LC light propagating along optical path 124 are combined by dichroic mirror 112 and passed to beam scanner 114.

Beam scanner 114 is configured to adjust the OCT measurement location on cornea 12 in two dimensions, such as the lateral dimension and the elevation dimension, in response to commands received from OCT engine 200 or, alternatively, control computer 300. Advantageously, beam scanner 114 scans the radiation (such as UV light) and the LC light together, while common optical path 120 ensures that the radiation and LC light travel coaxially and focus on the same location on cornea 12 in order to properly align the application and monitoring components of system 100. Generally, beam scanner 114 reflects (or deflects) the radiation, the LC light, and the reflected LC light along common optical path 120.

Beam scanner 114 may include a single mirror that rotates about two orthogonal axes to adjust the OCT measurement location on cornea 12 in two dimensions. Alternatively, beam scanner 114 may include two orthogonal mirrors, and each mirror independently rotates about an orthogonal axis to adjust the OCT measurement location on cornea 12 in two dimensions. The single mirror may be driven by a pair of electric motors or galvanometers (or galvos), while each orthogonal mirror may be driven by a single electric motor or galvanometer. Other drive systems are also contemplated, such as piezoelectric actuators, piezoelectric galvanometers, magnetorestrictive actuators, etc.

Alignment mirror 116 is configured to mechanically align cornea 12 to common optical path 120 of BDS 110. Generally alignment mirror 116 reflects or deflects the radiation (such as UV light), the LC light, and the reflected LC light 900 along common optical path 120. In certain embodiments, system 100 does not include alignment mirror 116, and common optical path 120 includes a straight optical path segment between beam scanner 114 and focusing lens 118.

Focusing lens 118 focuses the radiation (such as UV light) and the LC light traveling along common optical path 120 onto cornea 12, and focuses the reflected LC light from cornea 12 into common optical path 120.

In certain other embodiments, BDS 110 may be a fiber-based system in which certain free-space optical elements are replaced by optical fibers, electro-optical elements, etc. For example, optical fiber may replace at least certain portions of the free-space optical paths, such as portions of common optical path 120, optical path 122 and optical path 124, a fiber optic-based beam combiner may replace dichroic mirror 112, an electro-optical beam steering chip, such as an acoustic scanner, etc., an optical phased array (OPA) may replace beam scanner 114, etc.

FIG. 1B depicts a block diagram of system 100′ for corneal cross-linking with real-time monitoring, in accordance with embodiments of the present disclosure.

In certain embodiments, system 100′ includes, inter alia, beam delivery system (BDS) 110, CXL radiation source 130, OCT engine 200 and control computer 300. Generally, system 100′ includes the same components as system 100, with the exception of alignment mirror 116 and the exchange of the locations of CXL radiation source 130 and OCT engine 200 with respect to BDS 110. Additionally, common optical path 120 includes a straight optical path segment between beam scanner 114 and focusing lens 118.

FIG. 1C depicts a block diagram of system 100″ for corneal cross-linking with real-time monitoring, in accordance with embodiments of the present disclosure.

In certain embodiments, system 100′ includes, inter alia, beam delivery system (BDS) 110, CXL radiation source 130, OCT engine 200 and control computer 300. Generally, system 100″ is a simplified version of system 100′, includes the same components as system 100′, with the exception of dichroic mirror 112 and beam scanner 114.

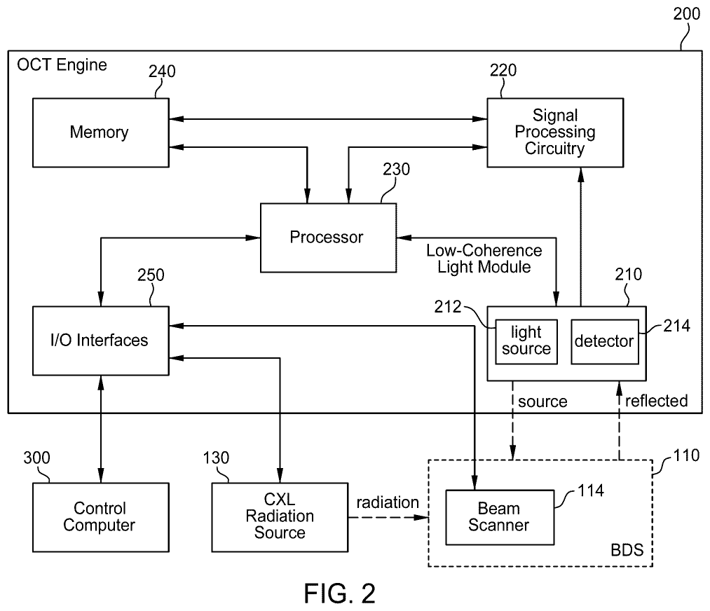

FIG. 2 depicts a block diagram of OCT engine 200, in accordance with embodiments of the present disclosure.

In certain embodiments, OCT engine 200 includes, inter alia, LC light module 210, signal processing circuitry 220, one or more processor(s) 230, storage element or memory 240, and I/O interfaces 250. LC light module 210 includes LC light source 212 and reflected LC light detector 214. Signal processing circuitry 220 may be coupled to LC light module 210 and memory 240, and processor 230 may be coupled to LC light module 210, signal processing circuitry 220, memory 240, and I/O interfaces 250. In certain embodiments, signal processing circuitry 220 is not present, and the functionality provided by signal processing circuitry 220 is provided by processor 230.

In certain embodiments, OCT engine 200 provides FD-OCT measurements using swept-source OCT (SS-OCT). In certain SS-OCT embodiments, LC light source 212 may be a swept-wavelength light source and reflected LC light detector 214 may be a high-speed photodetector. The swept-wavelength light source is configured to rapidly sweep a narrow line-width optical signal over a broad range of wavelengths during each A-scan, such as a Fourier Domain Mode Locked (FDML) laser with a 1050 nm center wavelength and a scan rate of 5 MHz or greater. Generally, the sweep rate of the swept-wavelength light source may be 100 kHz or greater. The high-speed photodetector is configured to sequentially detect the wavelength components of the reflected LC light signal (or interferometric signal) during one wavelength sweep (A-scan). In other words, reflected LC light detector 214 is configured to produce a spectral interferogram with fringe patterns during each wavelength sweep (A-scan). The spectral interferogram includes intensity data for each wavelength (or frequency) emitted by the swept-wavelength light source.

In other embodiments, OCT engine 200 provides FD-OCT measurements using spectral domain (SD-OCT). In certain SD-OCT embodiments, LC light source 212 may be a broadband optical source such as a superluminescent diode (SLD), and reflected LC light detector 214 may include a spectrometer and a high speed line camera that generates the spectral interferogram with fringe patterns during each A-scan.

A high-speed analog-to-digital (A/D) converter may be coupled to, or included within, LC light module 210 to convert the analog interferometric signal into a digital signal, which is generally known as a temporal OCT interferogram.

In certain embodiments, signal processing circuitry 220 is coupled to LC light module 210, and includes one or more microprocessors, application specific integrated circuits (ASICs), field programmable gate arrays (FPGAs), etc., configured to generate complex OCT data based on the temporal OCT interferogram received from reflected LC light detector 214. In certain embodiments, signal processing circuitry 220 includes a high-speed A/D converter (rather than LC light module 210), which converts the analog spectral interferogram signals received from reflected LC light detector 214 into OCT data.

Advantageously, the OCT data simultaneously includes intensity information for all of the depth layers in the A-scan at the OCT measurement location.

In certain embodiments, signal processing circuitry 220 may extract the intensity information for each depth layer in the A-scan by applying wavenumber remapping, dispersion compensation, Fourier transformation, etc., to the temporal OCT interferogram to generate complex OCT data. For example, signal processing circuitry 220 may apply a fast Fourier transform (FFT) to the temporal OCT interferogram to generate the complex OCT data. The amplitude of the complex OCT data may be squared to yield the intensity at different depths, while the phase of the complex OCT data may be further processed to provide extra information, as described below.

During a motion mode scan (also known as an M-mode scan), OCT engine 200 performs multiple A-scans at the same location, which generates a temporal OCT interferogram for that location. And, signal processing circuitry 220 may apply an FFT to the temporal OCT interferogram to generate temporal complex OCT data for that location.

Processor 230 may include one or more general-purpose or application-specific microprocessors, microcontrollers, etc., that execute instructions to perform control, computation, input/output, etc. functions for OCT engine 200. For example, processor 230 may be configured to synchronize the triggering of CXL radiation source 130, the scanning of beam scanner 114, and the measurement operations of LC light module 210 in order to acquire and send temporal OCT interferograms and complex OCT data to control computer 300. In certain other embodiments, CXL radiation source 130 and/or beam scanner 114 may be controlled by control computer 300.

Processor 230 may include a single integrated circuit, such as a micro-processing device, or multiple integrated circuit devices and/or circuit boards working in cooperation to accomplish the appropriate functionality. In certain embodiments, signal processing circuitry 220 is not present, and processor 230 may apply wavenumber remapping, dispersion compensation, Fourier transformation (such as an FFT), etc., to the temporal OCT interferogram to generate complex OCT data.

Generally, memory 240 may store instructions for execution by processor 230 as well as data, such as temporal OCT interferograms, complex OCT data, etc. Memory 240 may include a variety of non-transitory computer-readable medium that may be accessed by processor 230 as well as other components. In various embodiments, memory 240 may include volatile and nonvolatile medium, non-removable medium and/or removable medium. For example, memory 240 may include any combination of random access memory (RAM), dynamic RAM (DRAM), static RAM (SRAM), read only memory (ROM), flash memory, cache memory, and/or any other type of non-transitory computer-readable medium.

I/O interfaces 250 are configured to transmit and/or receive data from control computer 300, such as OCT measurement locations and complex OCT data, send commands to CXL radiation source 130 to emit radiation (such as UV light), send commands to beam scanner 114 to adjust the focus point of common optical path 120 to different OCT measurement locations on cornea 12, etc. Generally, data may be sent over wired and/or wireless connections. For example, I/O interfaces 370 may include one or more wired communications interfaces, such as USB, Ethernet, etc., and/or one or more wireless communications interfaces, coupled to one or more antennas, such as Bluetooth, WiFi, etc.

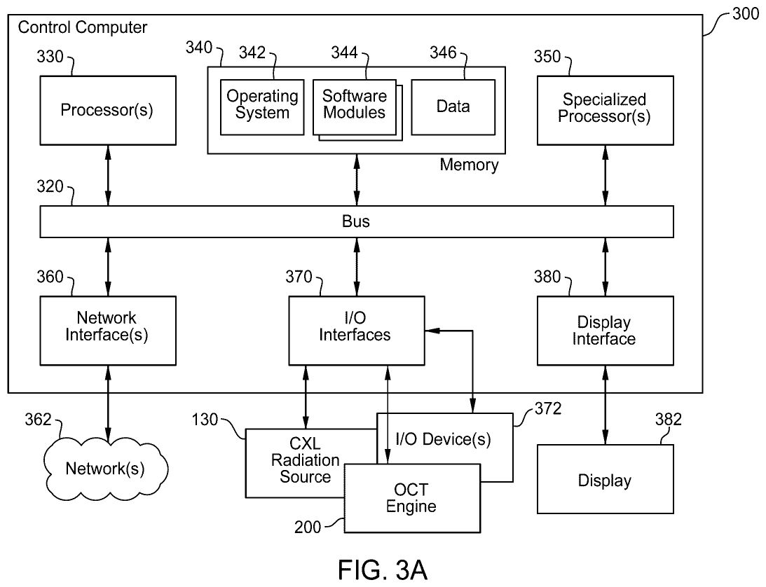

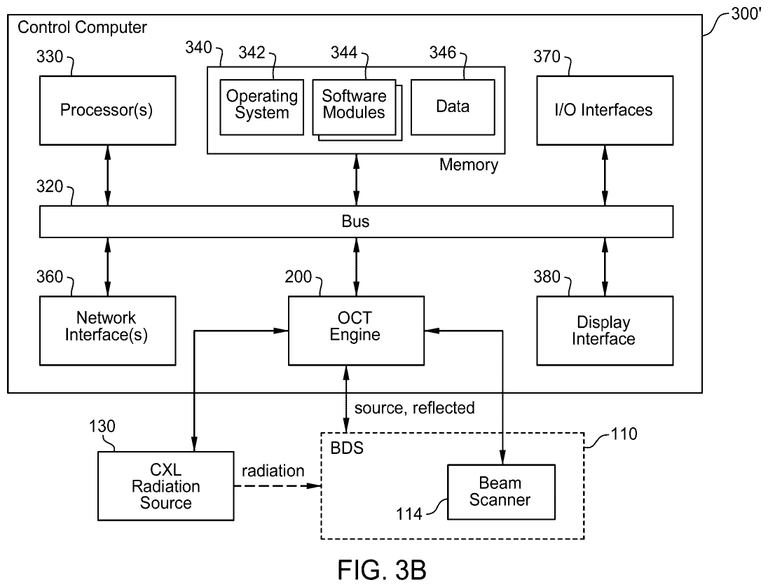

FIG. 3A depicts a block diagram of control computer 300, in accordance with embodiments of the present disclosure.

Control computer 300 includes bus 320 coupled to one or more processor(s) 330, storage element or memory 340, one or more network interface(s) 360, I/O interfaces 370, and display interface 380. In certain embodiments, control computer 300 also includes one or more specialized processor(s) or processing circuitry 350, such as, for example, graphics processing units (GPUs), ASICs, FPGAs, etc. Generally, network interface(s) 360 are coupled to one or more network(s) 362 using a wired or wireless connection, I/O interfaces 370 are coupled to one or more I/O device(s) 372 using a wired or wireless connection, and display interface 380 is typically coupled to display 382 using a wired connection.

In certain embodiments, CXL radiation source 130 and OCT engine 200 are coupled to I/O interfaces 370 using a wired or wireless connection, such as USB, Ethernet, Bluetooth, WiFi, etc. In certain embodiments, CXL radiation source 130 may send status information to control computer 300, over the connection, and control computer 300 may send configuration information, power commands, etc., to CXL radiation source 130 over the connection.

Bus 320 is a high-speed data transfer subsystem, such as a PCIe bus, etc., that transfers data between processor 330, memory 340, network interface(s) 360, I/O interfaces 370, and display interface 380. In certain embodiments, bus 320 also transfers data between these components and specialized processor or processing circuitry 350.

Processor 330 includes one or more general-purpose or application-specific microprocessors that execute instructions to perform control, computation, input/output, etc. functions for control computer 300. Each processor 330 may include a single integrated circuit, such as a micro-processing device, or multiple integrated circuit devices and/or circuit boards working in cooperation to accomplish the appropriate functionality. In addition, processor 330 may execute computer programs or modules, such as operating system 342, software modules 344, etc., stored within memory 340.

Generally, memory 340 stores instructions for execution by processor 330 as well as data. Memory 340 may include a variety of non-transitory computer-readable medium that may be accessed by processor 330 as well as other components. In various embodiments, memory 340 may include volatile and nonvolatile medium, non-removable medium and/or removable medium. For example, memory 340 may include any combination of random access memory (RAM), dynamic RAM (DRAM), static RAM (SRAM), read only memory (ROM), flash memory, cache memory, and/or any other type of non-transitory computer-readable medium.

Memory 340 stores various components for retrieving, presenting, modifying, and storing data, such as operating system 342, software modules 344, etc. Operating system 342 provides operating system functionality for control computer 300, while software modules 344 provide certain functionality when executed by processor 330. Data 346 may include data associated with operating system 342, software modules 344, etc.

Network interface(s) 360 is configured to transmit data to and from network(s) 362 using a wired and/or wireless connection. For example, network(s) 362 may include a local area network (LAN) that is connected to a wide area network (WAN) through a router, the WAN may be connected to the Internet through an Internet Service Provider (ISP), etc. Network(s) 362 may execute various network protocols, such as, for example, wired and/or wireless Ethernet, Bluetooth, etc. Network(s) 362 may also include various combinations of wired and/or wireless physical layers, such as, for example, copper wire or coaxial cable networks, fiber optic networks, WiFi networks, Bluetooth mesh networks, CDMA, FDMA and TDMA cellular networks, etc.

I/O interfaces 370 are configured to transmit and/or receive data from I/O devices 372. I/O interfaces 370 enable connectivity between processor 330, memory 340 and I/O devices 372 by encoding data to be sent from processor 330 or memory 340 to I/O devices 372, and decoding data received from I/O devices 372 for processor 330 or memory 340. Generally, data may be sent over wired and/or wireless connections. For example, I/O interfaces 370 may include one or more wired communications interfaces, such as USB, Ethernet, etc., and/or one or more wireless communications interfaces, coupled to one or more antennas, such as Bluetooth, WiFi, etc.

Generally, I/O devices 372 provide input to control computer 300 and/or output from control computer 300. As discussed above, I/O devices 372 are operably connected to control computer 300 using a wired and/or wireless connection. I/O devices 372 may include a local processor coupled to a communication interface that is configured to communicate with control computer 300 using the wired and/or wireless connection. For example, I/O devices 372 may include a touch screen, keyboard, mouse, touch pad, joystick, etc.

Display interface 380 is configured to transmit image data from control computer 300 to monitor or display 382.

FIG. 3B depicts a block diagram of control computer 300′, in accordance with embodiments of the present disclosure.

Control computer 300′ includes bus 320 coupled to one or more processor(s) 330, storage element or memory 340, one or more network interface(s) 360, I/O interfaces 370, and display interface 380, as described above with respect to control computer 300. In FIG. 3B, OCT engine 200 is depicted as a component of control computer 300′, such as one or more PCIe expansion boards, a PCIe expansion board coupled to one or more external modules, etc. In certain embodiments, OCT engine 200 may be housed within an external PCIe expansion system enclosure coupled to bus 320 via a PCIe connection.

To apply, monitor and adjust corneal cross-linking treatment in real-time using an integrated CXL and OCT system, such as system 100, 100′, 100″, control computer 300 determines a number of OCT measurement locations in a treatment region of cornea 12, and CXL radiation source 130 applies a corneal cross-linking treatment to the treatment region which may include irradiating a photosensitizer that has been applied to the treatment region of cornea 12.

During the application of the corneal cross-linking treatment by CXL radiation source 130, temporal OCT data are acquired at each OCT measurement location by OCT engine 200, temporal complex OCT data are generated by OCT engine 200 based on the temporal OCT data, biomechanical data are determined by control computer 300 based on the temporal complex OCT data received from OCT engine 200, and the corneal cross-linking treatment may be adjusted by control computer 300 based on the biomechanical data, such as increasing or decreasing the illumination intensity of the radiation (such as UV light) emitted by CXL radiation source 130, increasing or decreasing the time that the radiation is emitted by CXL radiation source 130 as compared to a treatment protocol threshold, etc.

Control computer 300 first divides at least one treatment region of cornea 12 into a number of OCT measurement locations which form a two-dimensional scan pattern, such as a symmetric or asymmetric scan pattern, a square or rectangular scan pattern, a circular or oval scan pattern, etc. In certain embodiments, two or more treatment regions may be identified, and the method for corneal cross-linking with real time monitoring may be repeated for each treatment region.

Generally, the diameter of cornea 12 varies between about 11.5 mm and 12.5 mm, with an average diameter of about 11.7 mm. In certain embodiments, the radiation is UV light that has a spot diameter of 4-11 mm, the LC light is near-infrared (IR) light that has a spot diameter of 10-20 μm, and the treatment region may have an area of about 65 mm², or, more generally, between 15 mm² and 100 mm². Generally, the size and number of the treatment regions may be determined by the UV light spot diameter, the extent of the desired corneal cross-linking treatment as a percentage of the area of cornea 12, and the diameter of cornea 12. Similarly, the number of OCT measurements for a treatment region may be determined based on the UV light spot diameter, the LC light spot diameter, and a degree of OCT measurement location overlap, such as 0%, 10%, 25%, etc., a degree of OCT measurement location spread or gap, such as 10%, 25%, etc., a measurement interval, such as 50 μm, 100 μm, etc. For example, for UV light with a spot diameter of 9 mm and LC light with a spot diameter of 20 μm, a single treatment region with a diameter of 9 mm may be divided into 8,000 OCT measurement locations with an interval of 100 μm between locations.

In order to acquire temporal OCT data in the treatment region, beam scanner 114 scans the LC light across the OCT measurement locations, which does not affect the irradiation of the photosensitizer by CXL radiation source.

Starting with the first OCT measurement location in the scan pattern, OCT engine 200 performs an M-mode scan at the OCT measurement location, which generates temporal OCT data for that location. The M-mode scan may require 5 ms, 10 ms, 15 ms, etc., to complete the scan, depending on the number of A-scans acquired. Signal processing circuitry 220 may then apply an FFT to the temporal OCT interferogram to generate temporal complex OCT data for that location. OCT engine 200 then commands beam scanner 114 to move to the next OCT measurement location in the scan pattern until temporal OCT data and temporal complex OCT data for all of the OCT measurement locations in the scan pattern have been acquired. In certain embodiments, control computer 300 may command beam scanner 114 to move to the OCT measurement locations in the scan pattern.

Generally, processor 330 (or specialized processor 350) may be configured to determine one or more corneal biomechanical properties at each OCT measurement location based on the complex-valued OCT data. For example, a time-dependent signal change rate, such as decorrelation, at each OCT measurement location may be used to determine tissue stiffness. The signal change rate is directly and inversely related to the degree of collagen confinement, which represents how tightly and densely the collagen fibrils are cross-linked. As new cross-links are formed between adjacent collagen fibrils during corneal cross-linking treatment, the corneal stiffness increases and the decorrelation coefficient decreases. Collagen confinement and corneal stiffness are biomechanical data that may be used to adjust the corneal cross-linking treatment.

In certain embodiments, the intensity of the radiation (such as UV light) emitted by CXL radiation source 130 may be increased or decreased based on the collagen confinement and corneal stiffness. For example, a rapid increase in collagen confinement and corneal stiffness may indicate that the corneal cross-linking treatment is proceeding too rapidly, and the intensity of the radiation (such as UV light) emitted by CXL radiation source 130 may be decreased. Similarly, a slow increase in collagen confinement and corneal stiffness may indicate that the corneal cross-linking treatment is proceeding too slowly, and the intensity of the radiation (such as UV light) emitted by CXL radiation source 130 may be increased.

In certain embodiments, the time that the corneal cross-linking treatment is applied, as compared to a treatment protocol threshold, may be increased or decreased based on the collagen confinement and corneal stiffness. For example, when the collagen confinement and corneal stiffness reach a stiffness threshold, then the radiation emitted by CXL radiation source 130 may be stopped, which may result in an increase or a decrease in the exposure time of the radiation emitted by CXL radiation source 130.

The use of other biomechanical data and control techniques are also contemplated.

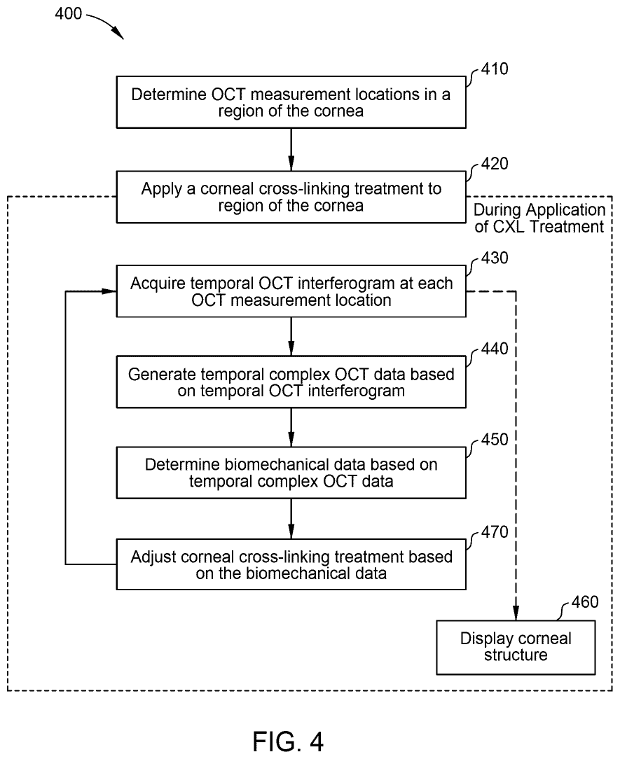

FIG. 4 depicts flow diagram 400 illustrating functionality associated with measuring corneal biomechanics, in accordance with embodiments of the present disclosure.

At 410, a plurality of OCT measurement locations in a region of a cornea are determined. Block 410 may be performed, for example, by control computer 300 or 300′. As discussed above, control computer 300 or 300′ may divide the treatment region into a number of OCT measurement locations which form a one dimensional or two-dimensional scan pattern for the region, such as a symmetric or asymmetric scan pattern, a square or rectangular scan pattern, a circular or oval scan pattern, etc. Flow continues to block 420.

At 420, a corneal cross-linking treatment is applied to the region of the cornea. Block 420 may be performed, for example, by CXL radiation source 130.

During the application of the corneal cross-linking treatment to the region of the cornea (i.e., block 420), blocks 430, 440 and 450 are periodically (or on demand) performed.

At 430, temporal OCT data at each OCT measurement location is acquired. Block 430 may be performed, for example, by OCT engine 200, as described above.

At 440, temporal complex OCT data is generated. Block 440 may be performed, for example, by OCT engine 200, as described above.

At 450, biomechanical data for the region of cornea 12 is determined based on the temporal complex OCT data. Block 450 may be performed, for example, by control computer 300 or 300′. As discussed above, the biomechanical data generated at each OCT measurement location in the region may include, for example, collagen confinement, corneal stiffness, etc.

At 460, the corneal cross-linking treatment may be adjusted based on the biomechanical data. Block 450 may be performed, for example, by control computer 300 or 300′. For example, an illumination intensity emitted by CXL radiation source 130 may be increased or decreased based on the biomechanical data, the exposure time of the radiation emitted by CXL radiation source 130 may be increased or decreased, etc.

In certain embodiments, at 470, a corneal structure may be displayed, by control computer 300 or 300′ on display 382, based on the OCT image.

The certain features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A method for corneal cross-linking with real-time monitoring, the method comprising:
determining a plurality of optical coherence tomography (OCT) measurement locations in a region of a cornea;
applying a corneal cross-linking treatment to the region of the cornea; and
during the applying of the corneal cross-linking treatment:
acquiring a temporal OCT interferogram at each OCT measurement location,
generating temporal complex OCT data based on the temporal OCT interferogram,
determining biomechanical data based on the temporal complex OCT data, and
adjusting the corneal cross-linking treatment based on the biomechanical data.

2. The method of claim 1, wherein the applying the corneal cross-linking treatment comprises:
irradiating, by a corneal cross-linking radiation source, a photosensitizer that has been applied to the region of the cornea; and

3. The method of claim 2, wherein the adjusting the corneal cross-linking treatment comprises:
increasing or decreasing an illumination intensity emitted by the corneal cross-linking radiation source based on the biomechanical data.

4. The method of claim 3, further comprising:
during the applying of the corneal cross-linking treatment:
displaying a corneal structure at each OCT measurement location.

5. The method of claim 1, wherein the biomechanical data comprise:
a collagen confinement; and
a corneal stiffness.

6. The method of claim 5, wherein:
the acquiring temporal OCT interferogram comprises acquiring M-mode OCT data at each OCT measurement location; and
the generating temporal complex OCT data comprises processing the temporal OCT interferogram based on a wavenumber remapping, a dispersion compensation, or a fast Fourier transform (FFT).

7. The method of claim 6, wherein:
the complex OCT data includes amplitude data and phase data; and
the determining the biomechanical data comprises:
determining a signal change rate based on the phase data of the temporal complex OCT data,
determining the collagen confinement based on the signal change rate, and
determining the corneal stiffness based on the collagen confinement.

8. The method of claim 6, wherein:
the complex OCT data includes amplitude data and phase data; and
the determining the biomechanical data comprises:
determining a signal change rate based on the amplitude data of the temporal complex OCT data,
determining the collagen confinement based on the signal change rate, and
determining the corneal stiffness based on the collagen confinement.

9. The method of claim 6, wherein:
the complex OCT data includes amplitude data and phase data; and
the determining the biomechanical data comprises:
determining a signal change rate based on the amplitude data and the phase data of the temporal complex OCT data,
determining the collagen confinement based on the signal change rate, and
determining the corneal stiffness based on the collagen confinement.

10. The method of claim 2, wherein:
the corneal cross-linking radiation source is configured to emit ultraviolet (UV) light that propagates along a common optical path to the cornea; and
the common optical path is defined by a beam delivery system (BDS).

11. The method of claim 10, wherein the acquiring temporal OCT interferogram comprises:
emitting, by a low-coherence (LC) light source, LC light that propagates along the common optical path to the OCT measurement location;
detecting, by a reflected LC light detector, reflected LC light that propagates along the common optical path from the OCT measurement location; and
generating, by a processor or signal processing circuitry coupled to the reflected LC light detector, the temporal OCT interferogram based on the reflected LC light.

12. The method of claim 11, wherein the BDS comprises a dichroic mirror, a beam scanner, and a focusing lens that define the common optical path.

13. The method of claim 12, wherein the dichroic mirror is configured to:
pass the UV light from the corneal cross-linking radiation source into the common optical path;
reflect the LC light from the LC light source into the common optical path; and
reflect the reflected LC light from the common optical path to the reflected LC light detector.

14. The method of claim 13, wherein the UV light and the LC light propagate coaxially along the common optical path to the OCT measurement location.

15. A system for corneal cross-linking with real-time monitoring, the system comprising:
a beam delivery system (BDS) defining a common optical path;
a corneal cross-linking radiation source configured to apply a corneal cross-linking treatment to a region of a cornea;
an optical coherence tomography (OCT) engine configured to:
acquire temporal OCT interferogram at each of a plurality of OCT measurement locations in the region via the common optical path, and
generate temporal complex OCT data based on the temporal OCT interferogram; and
a control computer, coupled to the OCT engine, the control computer comprising a processor configured to:
determine the plurality of OCT measurement locations in the region of the cornea,
send the OCT measurement locations to the OCT engine, and
during the corneal cross-linking treatment application:
receive temporal complex OCT data for each OCT measurement location from the OCT engine,
determine biomechanical data for the region of the cornea based on the temporal complex OCT data, and
adjust the corneal cross-linking radiation source based on the biomechanical data for the region of the cornea.

16. The system of claim 15, wherein:
the temporal OCT interferogram comprises M-mode OCT data; and
the processor being configured to generate temporal complex OCT data comprises the processor being configured to process the M-mode OCT data based on a wavenumber remapping, a dispersion compensation, or a fast Fourier transform (FFT).

17. The system of claim 16, wherein:
the complex OCT data includes amplitude data and phase data;
the biomechanical data for the region of the cornea comprise a collagen confinement and a corneal stiffness; and
the processor being configured to determine the biomechanical data comprises the processor being configured to:
determine a signal change rate based on the phase data of the temporal complex OCT data;
determine the collagen confinement based on the signal change rate; and
determine the corneal stiffness based on the collagen confinement.

18. The system of claim 17, wherein:
the processor being configured to apply the corneal cross-linking treatment comprises the processor being configured to emit ultraviolet light (UV) that propagates along the common optical path to the cornea; and
the processor being configured to adjust the corneal cross-linking radiation source comprises the processor being configured to increase or decrease a UV light illumination intensity emitted by the corneal cross-linking radiation source based on the biomechanical data.

19. The system of claim 18, wherein:
the OCT engine comprises a low-coherence (LC) light source, a reflected LC light detector, and a processor or signal processing circuitry configured to generate the temporal OCT interferogram and the temporal complex OCT data;
the LC light source is configured to emit LC light that propagates along the common optical path to the OCT measurement location; and
the reflected LC light detector is configured to detect reflected LC light that propagates along the common optical path from the OCT measurement location.

20. The system of claim 19, wherein the BDS comprises a dichroic mirror, a beam scanner, and a focusing lens that define the common optical path.
